(19) United States
(12) Patent Application Publication
(10) Pub. No.: US 2024/0115421 A1
(43) Pub. Date: Apr. 11, 2024

(54) REDUCING INTRAOCULAR PRESSURE (IOP) FLUCTUATIONS DURING PHACOEMULSIFICATION

(71) Applicant: JOHNSON & JOHNSON SURGICAL VISION, INC., Irvine, CA (US)

(72) Inventors: Assaf Govari, Haifa (IL); Vadim Gliner, Haifa (IL); Andres Claudio Altmann, Haifa (IL); Alon Boumendil, Givat Nili (IL)

(21) Appl. No.: 17/961,746

(22) Filed: Oct. 7, 2022

(51) Int. Cl. A61F 9/007

(52) U.S. Cl. CPC A61F 9/00745; A61M 2210/0612

(57) ABSTRACT

A phacoemulsification method includes specifying a target intraocular pressure (IOP) level to be maintained in an eye of a patient during a phacoemulsification procedure. A phacoemulsification probe is inserted into the eye. The eye is irrigated with irrigation fluid and material is aspired from the eye while aiming to maintain the target IOP level, including controlling IOP fluctuations by (i) when an actual IOP in the eye is in a defined high range, limiting the IOP fluctuations to a given value, and (ii) when the actual IOP is in a defined low range that is lower than the high range, allowing the IOP fluctuations to exceed the given value.

### FIELD OF THE DISCLOSURE

The present disclosure relates generally to phacoemulsification apparatuses and probes, and particularly to systems for control of IOP.

### BACKGROUND OF THE DISCLOSURE

A cataract is a clouding and hardening of the eye's natural lens, a structure which is positioned behind the cornea, iris and pupil. The lens is mostly made up of water and protein and as people age these proteins change and may begin to clump together obscuring portions of the lens. To correct this, a physician may recommend phacoemulsification cataract surgery. In the procedure, the surgeon makes a small incision in the sclera or cornea of the eye. Then a portion of the anterior surface of the lens capsule is removed to gain access to the cataract. The surgeon then uses a phacoemulsification probe, which has an ultrasonic handpiece with a needle. The tip of the needle vibrates at ultrasonic frequency to sculpt and emulsify the cataract while a pump aspirates particles and fluid from the eye through the tip. Aspirated fluids are replaced with irrigation of a balanced salt solution to maintain the anterior chamber of the eye. After removing the cataract with phacoemulsification, the softer outer lens cortex is removed with suction. An intraocular lens (IOL) is then introduced into the empty lens capsule restoring the patient's vision.

The present disclosure will be more fully understood from the following detailed description of the examples thereof, taken together with the drawings in which:

### BRIEF DESCRIPTION OF THE DRAWINGS

FIG. 1 is a schematic, pictorial view, along with an orthographic side view, of a phacoemulsification apparatus, in accordance with an example of the present disclosure;

FIG. 2 is a block diagram schematically describing an intraocular pressure (IOP) control scheme of the phacoemulsification apparatus of FIG. 1, in accordance with an example of the present disclosure; and

FIG. 3 is a flow chart schematically illustrating a method to control IOP while using the phacoemulsification apparatus of FIG. 1, in accordance with an example of the present disclosure.

### DETAILED DESCRIPTION OF EXAMPLES

### Overview

During phacoemulsification, irrigation fluid is flowed into the eye via an irrigation channel of a phacoemulsification probe, and in parallel emulsified lens particles are aspirated through an aspiration tip (e.g., a hollow needle) of the probe. To facilitate nominal and safe phacoemulsification operation, it is desired to maintain the intraocular pressure (IOP) at the surgical site at around an elevated value of 70 mmHg (while normal IOP is around 20 mmHg only). The IOP can be maintained by controlling the irrigation, the aspiration, or both. In some example embodiments, the IOP is monitored with a pressure sensor along the irrigation line, e.g., at or near the surgical site.

The desired working range of IOP during phacoemulsification is typically between 20 mmHg and 120 mmHg. Below 20 mmHg, the eye chamber may collapse. Above 120 mmHg, the eye may develop clouding. It is typically challenging to maintain a steady working IOP during a phacoemulsification procedure. The volume of the eye is small and even small fluctuations in pressure can significantly affect the IOP. In addition, suction of the cataract pieces is often accompanied by sharp changes in vacuum pressure through the tip. Although, the concurrent irrigation flow is operated to compensate for such fluctuations, the compensation provided is not always instantaneous.

Another important parameter in phacoemulsification procedures is followability. Followability is ability to attract cataract pieces to the aspiration tip in a steady consecutive manner. Followability may be compromised due to an increase irrigation flow that may push the cataract pieces away from the tip, e.g., in a circular motion around the aspiration tip.

Thus, even small flow variations in one of the pumps can result in noticeable fluctuations in IOP. These fluctuations may be distracting to the physician. For example, the eye chamber may noticeably expand and contract and/or fluid may periodically squirt out through the incision in the eye, both occurrences caused by IOP fluctuations. The fluctuations may expose the eye to IOP outside the aforementioned safe working range and potentially cause damage to the eye. In some cases, such fluctuations may compromise eye capsule stability. A proportional-integral-derivative (PID) controller may be used to regulate the IOP and reduce the fluctuations. To this end, a measured IOP is proportionally provided as negative feedback to the input signal of the PID controller. This mode of feedback is called hereinafter “nominal feedback.” Depending on how the PID parameters are defined, the PID controller may either reduce expected fluctuations in IOP at the expense compromised followability or improve followability at the expense of larger fluctuations in IOP.

According to some example embodiments, there is provided a control device and method to dynamically adjust the PID parameters based on an IOP that is currently detected in the surgical site. In some example embodiments, control device and method is configured to dynamically define the PID parameters to improve (or maximize) flowability at the expense of higher amplitude IOP fluctuations in a mid-range of IOP working pressure, e.g., 50 mmHg-90 mmHg, and dynamically switch to defining the PID parameters to reduce (or minimize) amplitude IOP fluctuations at the expense of reduced flowability at the upper and lower ranges of the working range, e.g., 20 mmHg-50 mmHg and 90 mmHg-120 mmHg. In some examples, changes in the PID parameters may be based on thresholds defining a high, medium, and low working range. In other examples, the PID parameters may be updated in a more continuous manner, e.g., for each IOP or group of IOPs computed.

Examples of the present disclosure that are described hereinafter provide methods and systems to optimize IOP that is regulated based on a physician's selected IOP working point (also called hereinafter target IOP level) and a processor calculating IOP tolerance (e.g., minimum and maximum IOP limits) according to the IOP target level. The processor aims to maintain the target IOP by, when the target IOP is in a high range or low range, limiting IOP fluctuations to a given low value, while when the target IOP is in a mid-range, allowing higher IOP fluctuations.

In some example embodiments, the control device includes a PID controller as well as feedback to the PID controller that dynamically updates the PID control parameters based on the current IOP of the surgical site.

### Apparatus Description

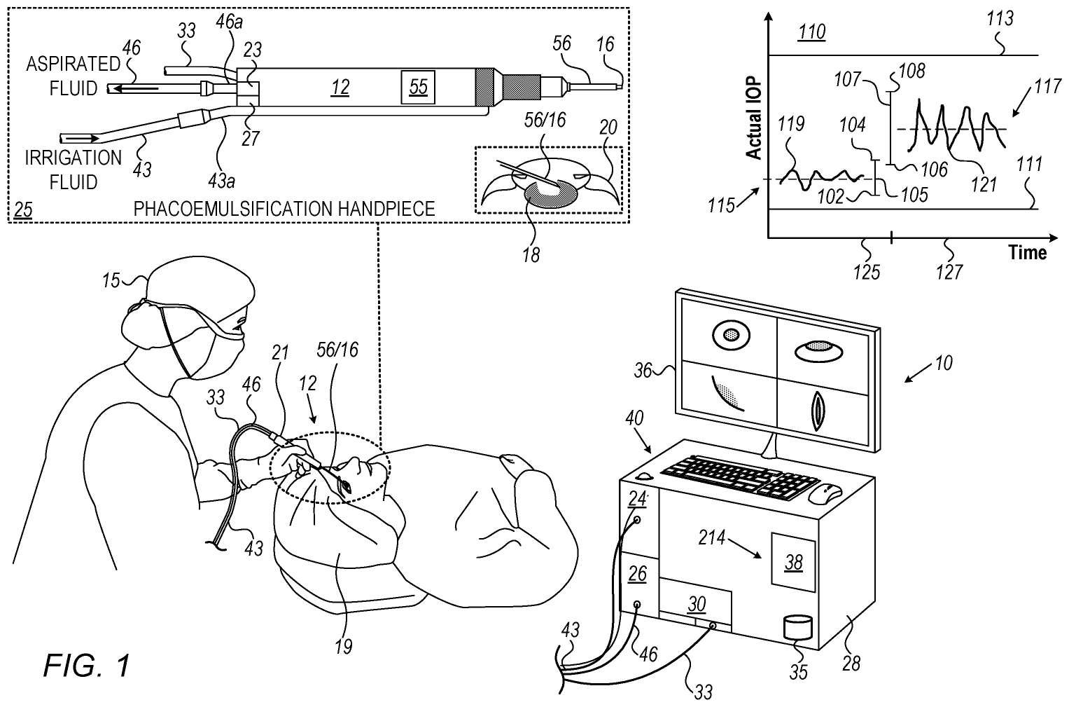

FIG. 1 is a schematic, pictorial view, along with an orthographic side view, of a phacoemulsification apparatus 10, in accordance with an example of the present disclosure.

As seen in the pictorial view of phacoemulsification apparatus 10, and in the orthographic side view inset 25, a phacoemulsification probe 12 (e.g., a handpiece) comprises a distal end 112 comprising a needle 16 and a coaxial irrigation sleeve 56 that at least partially surrounds needle 16 and creates a fluid pathway between the external wall of the needle and the internal wall of the irrigation sleeve, where needle 16 is hollow to provide an aspiration channel. Moreover, the irrigation sleeve may have one or more side ports at or near the distal end to allow irrigation fluid to flow toward the distal end of the handpiece through the fluid pathway and out of the port(s).

Needle 16 is configured for insertion into a lens capsule 18 of an eye 20 of a patient 19 by a physician 15 to remove a cataract. While the needle 16 (and irrigation sleeve 56) are shown in inset 25 as a straight object, any suitable needle may be used with phacoemulsification probe 12, for example, a curved or bent tip needle commercially available from Johnson & Johnson Surgical Vision, Inc., Irvine, CA, USA.

In the shown example, during the phacoemulsification procedure a irrigation pump 24, comprised in a console 28, pumps irrigation fluid from an irrigation reservoir (not shown) to the irrigation sleeve 56 to irrigate the eye. The fluid is pumped via an irrigation tubing line 43 running from console 28 to an irrigation channel 43a of probe 12. In another example, pump 24 may be coupled with or replaced by a gravity-fed irrigation source such as a balanced salt solution bottle/bag.

Eye fluid and waste matter (e.g., emulsified parts of the cataract) are aspirated via hollow needle 16 to a collection receptacle (not shown) by a processor-controlled aspiration pump 26, also comprised in console 28, using aspiration tubing line 46 running from aspiration channel 46a of probe 12 to console 28. In an example, a PID controller 214 module of processor 38 (e.g., a software module) controls an irrigation rate of irrigation pump 24 to maintain intraocular pressure (in case of sub-pressure indicated, for example, by sensor 27) within prespecified limits.

Channels 43a and 46a are coupled respectively with irrigation line 43 and aspiration line 46. Pumps 24 and 26 may be any pump known in the art (e.g., a peristaltic pump, progressive cavity pump). Using sensors (e.g., as indicated by sensors 27 and/or 23), controller 38 controls a flow rate of irrigation pump 24 and/or aspiration pump 26 to maintain intraocular pressure (IOP) within prespecified limits.

As noted above, controller 38 may control flow rate of irrigation pump 24 and/or aspiration pump 26, where one of the software modules running in processor 38 is a PID controller.

In the shown example, probe 12 includes an irrigation pressure sensor 27 coupled with irrigation channel 43a and an aspiration pressure sensor 23 coupled with an aspiration channel 46a. The control of irrigation pump 24 is done with a PID controller 214.

An example of the disclosed technique to control an actual IOP is given in graph 110. As described above, the allowable IOP working range during phacoemulsification is typically between a low IOP limit 111 (e.g., 20 mmHg) and a high IOP limit 113 (e.g., 120 mmHg). If physician 15 selects (e.g., during time duration 125) an IOP working point (i.e., target IOP level) at an IOP level 115 that is near the lower limit 111 of the working range (e.g., a low range 105), the disclosed technique defines the PID parameters to reduce fluctuations at the expense of reduced followability to avoid damaging the eye via sub-pressure.

If physician 15 selects (e.g., during time duration 127) an IOP working point (i.e., target IOP level) at an IOP level 117 that is in mid- to high-range (e.g., a high range 107), greater fluctuations 121 may be tolerated to provide better followability. To this end, the processor running the disclosed algorithm defines the PID parameters to improve the reaction time of the compensation.

In one example, using the real-time measured IOP, processor 38, or a processor of PID controller 214, estimates deviation of the measured IOP (e.g., a moving average IOP) from target IOP level and tolerance, and calculates a new target IOP level and/or new IOP tolerance. When the estimated deviation exceeds the allowed deviation and/or allowed tolerance (e.g., currently measured peak-to-peak IOP level in the eye), the PID controller applies a new target IOP level and/or the new tolerance.

In the shown example of FIG. 1, during phacoemulsification, PID controller module 214 of processor 38 controls the IOP by altering the irrigation flow in sleeve 56. Processor 38 estimates the IOP using readings from the irrigation pressure sensor 27 and an empirical offset, since the irrigation pressure is measured at the proximal end of handpiece 12.

Sensors 27 and 23 may be any sensor known in the art, including, but not limited to, a vacuum sensor or flow sensor. The sensor measurements (e.g., pressure, vacuum, and/or flow) are taken close to the proximal end of the handpiece where the irrigation outlet and the aspiration inlet are located, so as to provide processor 38 with an accurate indication of the actual measurements occurring within an eye and provide a short response time to a control loop comprised in processor 38.

A vacuum sensor, as discussed herein, includes types of pressure sensors that are configured to provide sufficiently accurate measurements of low sub-atmospheric pressures within a typical sub-pressure range at which aspiration is applied (e.g., between 1 mmHg and 650 mmHg). In an example, the same pressure sensor model is used to measure irrigation pressure and aspiration sub-pressure, using different sensor settings/calibrations.

As further shown, phacoemulsification probe 12 includes a piezoelectric crystal 55, coupled to a horn (not shown), that drives needle 16 to vibrate in a resonant vibration mode that is used to break a cataract into small pieces during a phacoemulsification procedure. Console 28 comprises a piezoelectric drive module 30, coupled with the piezoelectric crystal, using electrical wiring running in cable 33. Drive module 30 is controlled by a processor 38 that uses the drive signal or a small-amplitude monitoring signal (e.g., at a detuned frequency) via cable 33 to enable monitoring of an electrical impedance of crystal 55, which detects occlusions and performs preemptive steps, such as adjusting the irrigation rate and/or adjusting the aspiration rate to prevent a subsequent vacuum surge.

Processor 38 further conveys processor-controlled driving signals via cable 33 to, for example, maintain needle 16 at maximal vibration amplitude. The drive module may be realized in hardware or software, for example, in a proportional-integral-derivative (PID) control architecture.

Processor 38 may receive user-based commands via a user interface 40, which may include setting a vibration mode and/or frequency of the piezoelectric crystal, and setting or adjusting an irrigation and/or aspiration rate of the irrigation pump 24 and aspiration pump 26. Processor 38 may receive user-based commands via a user interface 40, which may include needle 16 stroke amplitude settings and turning on irrigation and/or aspiration. In an example, the physician uses a foot pedal (not shown) as a means of control. For example, foot pedal position one activates only irrigation, pedal position two activates both irrigation and aspiration, and pedal position three adds needle 16 vibration. Additionally, or alternatively, processor 38 may receive the user-based commands from controls located in a handle 21 of probe 12.

In an example, user interface 40 and display 36 may be integrated into a touch screen graphical user interface.

Some or all of the functions of processor 38 may be combined in a single physical component or, alternatively, implemented using multiple physical components. These physical components may comprise hard-wired or programmable devices, or a combination of the two. In some examples, at least some of the functions of processor 38 may be carried out by suitable software stored in a memory 35 (as shown in FIG. 1). This software may be downloaded to a device in electronic form, over a network, for example. Alternatively, or additionally, the software may be stored in tangible, non-transitory computer-readable storage media, such as optical, magnetic, or electronic memory.

The apparatus shown in FIG. 1 is simplified for clarity of presentation. For example, the disclosed PID control scheme may be applied to other control devices of the system, such as valves and/or the irrigation pump.

### Control Scheme for Reducing IOP Fluctuations During Phacoemulsification

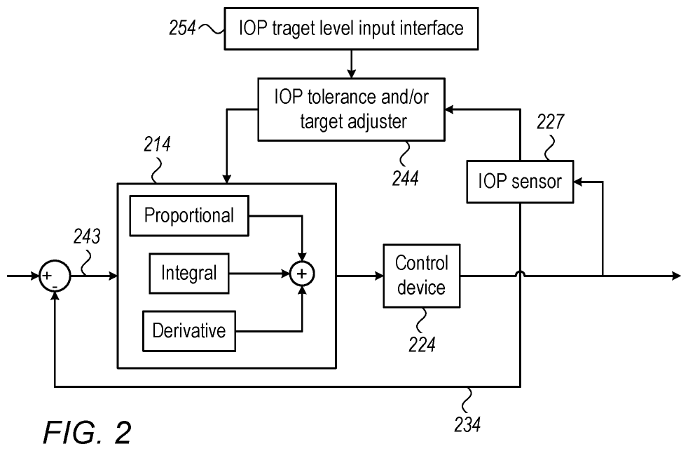

FIG. 2 is a block diagram schematically describing an intraocular pressure (IOP) control scheme of the phacoemulsification apparatus 10 of FIG. 1, in accordance with an example of the present disclosure.

As seen in the disclosed example, PID controller 214 has a nominal feedback loop 234 over the controller, which provides nominal control of a control device 224, such as of irrigation pump 24 seen in FIG. 1. The feedback signal is proportional to the actual IOP, and this quantity is subtracted at the input 243 to provide negative feedback.

As further seen, there is an additional disclosed secondary feedback loop 244, on top of the nominal control scheme of loop 234. A user can set an initial target IOP level (such as level 115) via an interface 254. The disclosed technique achieves the aforementioned adjustments (e.g., shifting target level and/or control of size of IOP fluctuations) by the secondary feedback loop 244 on the PID controller adjusting one or more of the PID parameters based on the actual IOP measured in real time by sensor 227 (which may be sensor 27 of FIG. 1).

The adjustment triggered by secondary loop 244 may include the PID controller shifting a selected IOP level (e.g., one of levels 115 and 117) and/or changing tolerance (e.g., narrowing it) based, for example, on a currently measured peak-to-peak IOP level in the eye (e.g., measured fluctuations 119 or 121). Another possible adjustment may be shifting the IOP target value, as described above.

The example block diagram shown in FIG. 2 is highly simplified and chosen purely for the sake of conceptual clarity. A typical system may include, for example, a feedback loop on an additional control device such as aspiration pump 26. These and other details of an actual system, such as a pressure sensor providing readings required for the control scheme, are omitted for simplicity of presentation.

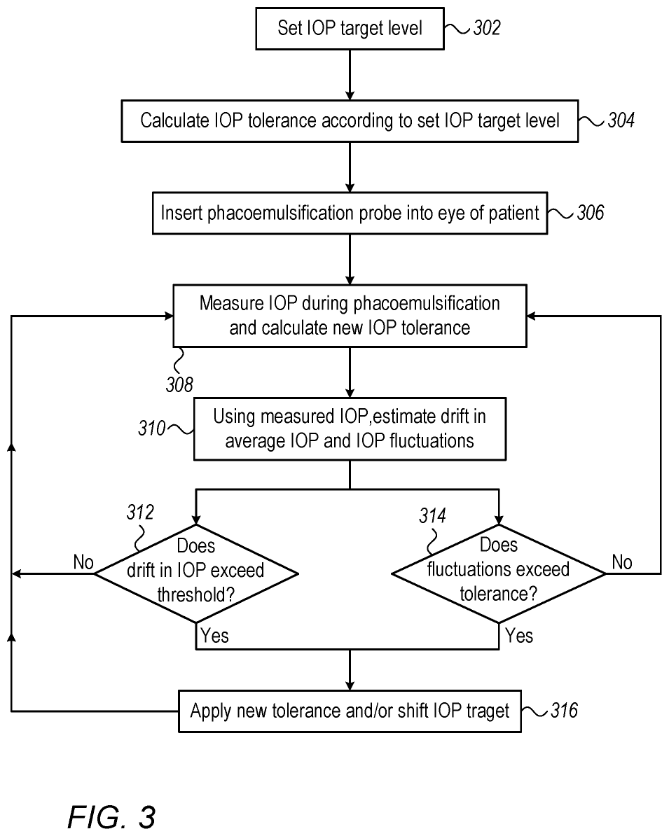

FIG. 3 is a flow chart schematically illustrating a method for controlling IOP while using the phacoemulsification apparatus 10 of FIGS. 1 and 2, in accordance with an example of the present disclosure. The process begins with physician 15 setting target IOP level (e.g., level 115), via interface 254, at target IOP level inputting step 302.

Subsequently, a processor of the system calculates IOP tolerance (e.g., tolerance 105) and values (e.g., values 102 and 104) according to the set target IOP level, at tolerance calculation step 304.

At probe insertion step 306, physician 15 inserts a phacoemulsification probe (e.g., needle 16 with sleeve 56) into an eye of a patient.

The physician operates the system by irrigating the eye with irrigation fluid and aspiring material from the eye, at phacoemulsification step 308, during which a sensor such as sensor 227 measures the IOP.

At IOP monitoring step 310, using the real-time measured IOP (during phacoemulsification), the processor recalculates the tolerance and repeatedly estimates shifts in target IOP level that may be required.

If the processor identifies a deviation in IOP, such as a drift in average IOP or fluctuations increased above tolerance, the processor applies recalculated tolerance and/or shifted value to reconfigure the controller of IOP to improve IOP control.

### EXAMPLES

### Example 1

A phacoemulsification method includes specifying a target intraocular pressure (IOP) level to be maintained in an eye (20) of a patient during a phacoemulsification procedure. A phacoemulsification probe (12) is inserted into the eye (20). The eye is irrigated with irrigation fluid and material is aspired from the eye while aiming to maintain the target IOP level, including controlling IOP fluctuations by (i) when an actual IOP in the eye is in a defined low range (105), limiting the IOP fluctuations to a given value, and (ii) when the actual IOP is in a defined high range (107) that is higher than the low range, allowing the IOP fluctuations to exceed the given value.

### Example 2

The method according to example 1, wherein controlling the IOP fluctuations includes the steps of: repeatedly measuring the actual IOP; using the repeatedly measured actual IOP, estimating a deviation from one or both of (i) the target IOP level (115, 117) and (ii) a target IOP tolerance (105, 107); when the estimated deviation exceeds an allowed deviation, calculating at least one of a new target IOP tolerance and a new target IOP level, and applying at least one of the new target IOP level and the new tolerance.

### Example 3

The method according to example 2, wherein the allowed deviation (105, 107) is a moving average of a difference between the measured actual IOP and the target IOP level (115, 117).

### Example 4

The method according to any of examples 2 and 3, wherein calculating the new target IOP tolerance comprises narrowing the target IOP tolerance (105, 107).

### Example 5

The method according to any of examples 2 and 3, wherein calculating the new target IOP tolerance comprises calculating an asymmetric IOP tolerance with respect to the target IOP level (115, 117).

### Example 6

The method according to any of example 2 through 5, wherein applying at least one of the new target IOP level and the new target IOP tolerance comprises reconfiguring control parameters of a rate of irrigation.

### Example 7

The method according to any of example 2 through 5, wherein applying at least one of the new target IOP level and the new target IOP tolerance comprises reconfiguring control parameters of a rate of aspiration.

### Example 8

The method according to any of example 2 through 7, wherein applying at least one of the new target IOP level and the new target IOP tolerance comprises reconfiguring control parameters of a PID controller (214) that controls irrigation or aspiration.

### Example 9

The method according to any of example 2 through 8, wherein controlling the IOP fluctuations comprises repeatedly adapting the target IOP level (115, 117) and the target IOP tolerance (105, 107) in real time with a given repetition rate.

### Example 10

A phacoemulsification system includes an interface (254) and a controller (214). The interface is configured for specifying a target intraocular pressure (IOP) level (115, 117) to be maintained in an eye of a patient during a phacoemulsification procedure. The controller is configured to, during irrigating the eye (20) with irrigation fluid and aspiring material from the eye using a phacoemulsification probe (12) inserted into the eye, aim to maintain the target IOP level, including controlling IOP fluctuations by (i) when an actual IOP in the eye is in a defined low range (105), limiting the IOP fluctuations to a given value, and (ii) when the actual IOP is in a defined high range (107) that is higher than the low range, allowing the IOP fluctuations to exceed the given value.

It will be appreciated that the examples described above are cited by way of example, and that the present disclosure is not limited to what has been particularly shown and described hereinabove. Rather, the scope of the present disclosure includes both combinations and sub-combinations of the various features described hereinabove, as well as variations and modifications thereof which would occur to persons skilled in the art upon reading the foregoing description and which are not disclosed in the prior art. Documents incorporated by reference in the present patent application are to be considered an integral part of the application except that to the extent any terms are defined in these incorporated documents in a manner that conflicts with the definitions made explicitly or implicitly in the present specification, only the definitions in the present specification should be considered.

1. A phacoemulsification method, comprising:
specifying a target intraocular pressure (IOP) level to be maintained in an eye of a patient during a phacoemulsification procedure;
inserting a phacoemulsification probe into the eye; and
irrigating the eye with irrigation fluid and aspiring material from the eye while aiming to maintain the target IOP level, including controlling IOP fluctuations by (i) when an actual IOP in the eye is in a defined low range, limiting the IOP fluctuations to a given value, and (ii) when the actual IOP is in a defined high range that is higher than the low range, allowing the IOP fluctuations to exceed the given value.

2. The method according to claim 1, wherein controlling the IOP fluctuations comprises the steps of:
repeatedly measuring the actual IOP;
using the repeatedly measured actual IOP, estimating a deviation from one or both of (i) the target IOP level and (ii) a target IOP tolerance;
when the estimated deviation exceeds an allowed deviation, calculating at least one of a new target IOP tolerance and a new target IOP level, and applying at least one of the new target IOP level and the new tolerance.

3. The method according to claim 2, wherein the allowed deviation is a moving average of a difference between the measured actual IOP and the target IOP level.

4. The method according to claim 2, wherein calculating the new target IOP tolerance comprises narrowing the target IOP tolerance.

5. The method according to claim 2, wherein calculating the new target IOP tolerance comprises calculating an asymmetric IOP tolerance with respect to the target IOP level.

6. The method according to claim 2, wherein applying at least one of the new target IOP level and the new target IOP tolerance comprises reconfiguring control parameters of a rate of irrigation.

7. The method according to claim 2, wherein applying at least one of the new target IOP level and the new target IOP tolerance comprises reconfiguring control parameters of a rate of aspiration.

8. The method according to claim 2, wherein applying at least one of the new target IOP level and the new target IOP tolerance comprises reconfiguring control parameters of a PID controller that controls irrigation or aspiration.

9. The method according to claim 2, wherein controlling the IOP fluctuations comprises repeatedly adapting the target IOP level and the target IOP tolerance in real time with a given repetition rate.

10. A phacoemulsification system, comprising:
an interface configured for specifying a target intraocular pressure (IOP) level to be maintained in an eye of a patient during a phacoemulsification procedure; and
a controller configured to, during irrigating the eye with irrigation fluid and aspiring material from the eye using a phacoemulsification probe inserted into the eye, aim, to maintain the target IOP level, including controlling IOP fluctuations by (i) when an actual IOP in the eye is in a defined low range, limiting the IOP fluctuations to a given value, and (ii) when the actual IOP is in a defined high range that is higher than the low range, allowing the IOP fluctuations to exceed the given value.

11. The system according to claim 10, wherein the controller is configured to control the IOP fluctuations by performing the steps of:
repeatedly measuring the actual IOP;
using the repeatedly measured actual IOP, estimating a deviation from one or both of (i) the target IOP level and (ii) a target IOP tolerance;
when the estimated deviation exceeds an allowed deviation, calculating at least one of a new target IOP tolerance and a new target IOP level, and applying at least one of the new target IOP level and the new tolerance.

12. The system according to claim 11, wherein the allowed deviation is a moving average of a difference between the measured actual IOP and the target IOP level.

13. The system according to claim 11, wherein the controller is configured to calculate the new target IOP tolerance by narrowing the target IOP tolerance.

14. The system according to claim 11, wherein the controller is configured to calculate the new target IOP tolerance by calculating an asymmetric IOP tolerance with respect to the target IOP level.

15. The system according to claim 11, wherein the controller is configured to apply at least one of the new target IOP level and the new target IOP tolerance by reconfiguring control parameters of a rate of irrigation.

16. The system according to claim 11, wherein the controller is configured to apply at least one of the new target IOP level and the new target IOP tolerance by reconfiguring control parameters of a rate of aspiration.

17. The system according to claim 11, wherein the controller is configured to apply at least one of the new target IOP level and the new target IOP tolerance by reconfiguring control parameters of a PID controller that controls irrigation or aspiration.

18. The system according to claim 11, wherein the controller is configured to control the IOP fluctuations by repeatedly adapting the target IOP level and the target IOP tolerance in real time with a given repetition rate.
